(19) United States
(12) Patent Application Publication
(10) Pub. No.: US 2002/0037829 A1
(43) Pub. Date: Mar. 28, 2002

(54) Use of DPPIV inhibitors as diuretic and anti-hypertensive agents

(76) Inventors: Peter S. Aronson, Guilford, CT (US); Adriana Girardi, S?atilde;o Paulo (BR); Felix Knauf, Berlin (DE)

Correspondence Address: MARY M. KRINSKY, Ph. D., J.D., PATENT ATTORNEY, 79 TRUMBULL STREET, NEW HAVEN, CT, 06511, US

(21) Appl. No.: 09/938,325

(22) Filed: Aug. 23, 2001

Related U.S. Application Data
(60) Provisional application No. 60/227,400, filed on Aug. 23, 2000.

(51) Int. Cl. A61K 31/00; C12N 9/99

(52) U.S. Cl. 514/1; 435/184

(57) ABSTRACT

Dipeptidyl peptidase IV inhibitors are used as diuretics and anti-hypertensive agents.

### CROSS-REFERENCES TO RELATED APPLICATIONS

This application claims priority benefit of provisional application U.S. Ser. No. 60/227,400, filed Aug. 23, 2000.

### STATEMENT REGARDING FEDERALLY SPONSORED RESEARCH

The invention was made with partial government support under NIH grant DK-33793. The government has certain rights in the invention.

### BACKGROUND OF THE INVENTION

1. Field of the Invention

This invention relates to a new class of diuretics and antihypertensive agents and methods for their use.

2. Description of Related Art

Epidemiologic studies have clearly demonstrated that elevated blood pressure is correlated with an increased incidence of cardiovascular disease, including stroke, renal failure, congestive heart failure, and myocardial infarction. The prevalence of hypertension increases with age in all groups: blacks, whites, men, and women. Hypertension is an extremely common health problem in the geriatric population, afflicting approximately 65% of persons in the 65- to 74-year-old group (Bennett, J.C., and Plum, F., eds., Cecil's Textbook of Medicine, 20th ed., W. B. Saunders Co., Philadelphia, 1996, p. 258; this reference and others cited herein are expressly incorporated in their entireties by reference). Blacks have a higher prevalence of hypertension than whites, and men have a higher overall prevalence than women (ibid.).

There is a great variety of drugs available for use in treating hypertension, including thiazide, loop, and potassium-sparing diuretics, β-adrenergic receptor blocking agents, calcium channel blockers, and angiotensin-converting enzyme inhibitors. Unfortunately, the adverse metabolic effects of some classes of antihypertensive drugs may increase coronary risk and offset the benefit of blood pressure reduction. Hence, there is considerable interest in developing new drugs, particularly those that act specifically on the regulation of renal sodium chloride (NaCl) excretion, which is responsible for maintaining NaCl balance and long-term control of blood pressure.

### BRIEF SUMMARY OF THE INVENTION

It is the primary objective of this invention to provide a new class of diuretics that are agents useful for treating hypertension.

It is a further and more specific objective of the invention to provide therapeutic agents for inhibiting renal sodium absorption.

These and other objectives are achieved by the present invention, which provides methods for treating hypertensive patients by administering to them an effective amount of at least one dipeptidyl peptidase IV (abbreviated herein as “DPP IV”) inhibitor. This is typically achieved by administering a pharmaceutical composition containing a DPP IV inhibitor. Exemplary inhibitors include peptides, acyl pyrrolidides and thiazolidides that are specific in their inhibition of DPP IV, stable, and non-toxic, such as those described by ProBioDrug at www.probiodrug.de, who develop and market inhibitors for therapeutic use, and described in the scientific literature (e.g., in Stocker, et al., Diabetes, 50: A522 (2001), Freyse, E. J., et al., Diabetes 50: A514 (2001), Pospisilik, J. A., et al., Diabetes 50: A311 (2001), Pederson, R. A., et al., Diabetes 47: 1253 (1998), and Hildebrandt, M., et al., Immunology 53: 449 (2001)). The use of the tripeptide Ile-Pro-Ile, also called diprotin A, is illustrated hereafter.

### BRIEF DESCRIPTION OF THE INVENTION

This invention is based upon the finding that dipeptidyl peptidase IV inhibitors can be used to regulate renal sodium transport and have efficacy for the long term control of blood pressure.

### EXAMPLE

The following example is presented to further illustrate and explain the present invention and should not be taken as limiting in any regard.

The great majority of NaCl filtered by the kidney is reabsorbed in the first part of the nephron, the proximal tubule. In the proximal tubule, Na+—H+ exchanger isoform NHE3 is the most important pathway for mediating Na+ reabsorption.

In an attempt to identify proteins that assemble with NHE3, monoclonal antibodies (mAbs) against affinity-purified NHE3 protein complexes isolated from solubilized rabbit renal brush border membranes were generated. Hybridomas were selected based on ability to immunoprecipitate NHE3. One of these antibodies, 1D11, labeled a 110 kDa protein, but not monomeric NHE3 (80 kDa) in immunoblotting experiments. By immunofluorescence microscopy, 1D11 stained the brush border membrane of proximal tubule cells. To test if the “1D11 protein” is specifically associated with NHE3, immunoprecipitations were carried out using either the low speed (15,000× g for 10 min) or high speed (200,000× g for 1 hr) supernatants from Triton X-100 solubilized renal brush border membranes. MAb 1D11 co-precipitated NHE3 but not the microvillar protein villin from both low and high speed supernatants.

Having demonstrated that the 1D11 protein and NHE3 are truly associated, immunoaffinity chromatography was used to isolate the protein against which mAb 1D11 is directed. The 1D11 antibody was immobilized on Sepharose CL-4B beads, and then the 1D11 protein was purified from solubilized brush border membranes. N-terminal sequencing of the purified 1D11 protein identified it as dipeptidylpeptidase IV (DPPIV) (EC 3.4.14.5). Finally, to confirm that the 1D11 protein is DPPIV, a specific enzymatic assay for DPPIV was performed. It was found that DPPIV enzymatic activity was virtually completely removed from solubilized rabbit brush border membranes passed through the same 1D11 affinity column, and was recovered in the eluted fractions.

Taken together, these experiments revealed an unexpected association of the brush border Na+—H+ exchanger NHE3 with dipeptidylpeptidase IV in the proximal tubule. This work describing the association of NHE3 with DPPIV was published as an abstract (Girardi, A. C. C., et al., J. Am. Soc. Nephrol. 10:4A, 1999.)

As an initial approach to examine the physiological role of the association of DPPIV with NHE3, Diprotin A, a specific competitive inhibitor that binds to the active site of DPPIV, affects NHE3 activity in OKP cells was studied. OKP is a line of opossum proximal tubule cells that has transport properties very similar to the native proximal tubule and is therefore commonly used as an in vitro model for the mammalian proximal tubule.

The concentration dependence for Diprotin A inhibition of DPPIV activity was first evaluated. A calorimetric enzyme assay using glycylproline p-nitroanilide tosylate as substrate showed that activity of DPPIV in OKP cells is completely inhibited by 1 mM Diprotin A. ²²Na uptake assays were performed in 24 well plates in which OKP cells were pre-incubated for 20 minutes in a NH3/NH4+ buffer, pH 7.4, in the presence or absence of 1 mM Diprotin A. NH₃/NH₄⁺ buffer was then removed and cells were incubated with a NH₃/NH₄⁺ free solution containing ²²Na and either vehicle or 1 mM Diprotin A. It was confirmed that Na⁺ influx measured under these conditions is ethylisopropylamiloride (EIPA)-sensitive and HOE-694 resistant, consistent with NHE3 activity. Diprotin A inhibited EIPA-sensitive Na/H exchange activity by 36.7±5.3%. To test the specificity of this inhibition, activity of another brush border transport process, Cl⁻-formate exchange, measured as Cl⁻ gradient-stimulated, DIDS-sensitive uptake of 14C-formate was assayed. There was no significant effect of Diprotin A on Cl⁻ formate exchange activity.

It was concluded from these studies that the DPPIV inhibitor Diprotin A decreases NHE3 activity in OKP cells. These results suggest that the association of DPPIV with NHE3 in oligomeric complexes may be involved in regulation of NHE3 activity. Moreover, these studies suggested that DPPIV inhibitors may be useful therapeutic agents for inhibiting renal Na+ reabsorption in the proximal tubule and therefore may be of use as diuretic and anti-hypertensive agents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

1. A method for treating hypertension in a patient in need thereof comprising administering to the patient an effective amount of a dipeptidyl peptidase IV inhibitor.
